(19) United States
(12) Patent Application Publication
(10) Pub. No.: US 2025/0244316 A1
(43) Pub. Date: Jul. 31, 2025

(54) LABEL AND MARKER FOR MARKING A TARGET MOLECULE IN A BIOLOGICAL SAMPLE

(71) Applicant: Leica Microsystems CMS GmbH, Wetzlar (DE)

(72) Inventors: Soeren ALSHEIMER, Wetzlar (DE); Arnold GISKE, Wetzlar (DE)

(21) Appl. No.: 18/855,645

(22) PCT Filed: Apr. 14, 2022
(86) PCT No.: PCT/EP2022/060035

§ 371 Date: Oct. 10, 2024

(51) Int. Cl. G01N 33/532; B82Y 15/00; B82Y 35/00; G01N 21/64

(52) U.S. Cl. CPC G01N 33/532; G01N 21/6428; B82Y 15/00; B82Y 35/00; G01N 2021/6439; G01N 2458/10

(57) ABSTRACT

A label for usage in a marker for marking a target molecule in a biological sample includes a nanostructure backbone, and at least one group of dyes comprising a first dye and a second dye attached to the nanostructure backbone. The first dye and the second dye are within a distance from each other that enables an energy transfer between the first dye and the second dye. One of the dyes of the group of dyes is configured to emit light upon the energy transfer.

### CROSS REFERENCE TO RELATED APPLICATIONS

This application is a U.S. National Phase application under 35 U.S.C. § 371 of International Application No. PCT/EP2022/060035, filed on Apr. 14, 2022. The International Application was published in English on Oct. 19, 2023 as WO 2023/198291 A1 under PCT Article 21 (2).

### FIELD

Embodiments of the present invention relate to a label for usage in a marker for marking a target molecule in a biological sample, and to a marker for marking a target molecule in a biological sample.

### BACKGROUND

In order to address key problems in the field of life sciences it is vital to precisely detect the presence of an analyte or molecular target in one of a biological sample (e.g. tissue samples or cell cultures), an environmental sample (a soil sample), a water sample, a diagnostic procedure (e.g. in a solid or liquid biopsy or a sample prepared from either) or a lysate or extract from such a sample. This can be done by introducing markers into the sample that bind to specific structures, e.g. specific biomolecules. These markers typically comprise an affinity reagent that attaches to the structure in question and a label with a fluorescent dye that is either directly conjugated to the affinity reagent or attached to the affinity reagent by means of a secondary affinity reagent. There are various techniques for analysing biological samples prepared in this way.

The plexing level in fluorescence microscopy, i.e. the number of different fluorescent dyes that can be read out at the same time is generally low and in the case of fluorescence microscopy typically in the range of 1 to 5 dyes for channel-based readouts and 5 to 12 dyes for readouts with spectral detectors, which use dispersive optical elements, such as prisms or gratings in combination with multiple detectors or array detectors. In fluorescence-based cytometry and sorting, somewhat higher plexing levels have been attained, but also in this case, plexing is limited to a low number of dyes and consequently markers that can be readout in one experiment.

“Fluorescent cell barcoding” is a multiplexing technique developed by Krutzig and Nolan 2006 and is based on using different mixtures of three fluorescent dyes as described in Nat Methods. 2006 May; 3 (5): 361-8. doi: 10.1038/nmeth872.

Citing from Tsai et al. 2020 “Fluorescent cell barcoding (FCB) is a multiplexing technique for high-throughput flow cytometry (FCM). Although powerful in minimizing staining variability, it remains a subjective FCM technique because of inter-operator variability and differences in data analysis” (J Immunol Methods 2020 February; 477:112667.doi: 10.1016/j.jim.2019.112667. Epub 2019 Nov. 11.) Both the subjectivelness of the technique and inter-operator variability of this method are inherently related to the fact it is based on encoding a part of the information in the hues of dyes, i.e. in intensity variations like in for example light green, green, dark-green, which severely limits the use of this technique.

Rare cells, like e.g. adult stem cells, circulating tumour cells and reactive immune cells (e.g. T-Cells, B-Cells, or NK-cells reactive to a certain antigen), are of great interest to basic and translational researchers. Reactive immune cells such as B-cell clones for instance that react to a certain pathogen, e.g. a virus, can produce antibodies against a particular pathogen and are of great value to generate urgently needed therapeutic antibodies. Similarly, reactive T-cells are sought after in the context of personalised medicine and the treatment of cancer and other diseases. Once a reactive T-cell is identified and isolated, the genetic sequence encoding the corresponding T-cell receptor displaying affinity against the target antigen can be cloned and used to generate genetically engineered T-cells such as CAR-T cells. Similarly, circulating tumour cells are expected to have great value for diagnosing cancer, predicting diseases outcomes, managing therapies, and for the discovery of new cancer drugs and cell-based therapeutics. Suspensions of cells containing rare cells are typically derived from either a tissue sample by means of dissociation or from a liquid biopsy. The identification, analysis, and isolation of rare cells in these samples, in particular the analysis of these cells on the single cell level (single cell analysis, SCA) is therefore of great value for basic and translational research, diagnostic and therapeutic applications as well as in the context of bioprocessing and development and manufacturing of biologics and cellular therapeutics.

As the ability to identify and differentiate diverse cell types expands, the identification of cell types becomes more granular, i.e. the rare cell populations of interest are smaller and better defined. Thus, in order to find rare cells of interest a high (<100 k), very high (<1M), or ultra-high (>1M) number of cells typically needs to be analysed.

Bright, photostable labels or reporters or tags, which allow fast readout on different fluorescence readout devices like widefield microscopes, confocal microscopes, cytometers or fluorescence activated cell sorters are therefore sought after. Ideally these labels are also small and can be reliably discerned from each other by the readout device. Labels may be built on a variety of backbone structures as reviewed in Gungun Lin et al. 2018 which cover a broad range of sizes (Chem 4, 997-1021 May 10, 2018: “The quest for optical multiplexing in bio-discoveries.”).

Thus, in order to mark and identify a large number of different cell types a correspondingly large set of labels and corresponding markers is required.

### SUMMARY

Embodiments of the present invention provide a label for usage in a marker for marking a target molecule in a biological sample. The label includes a nanostructure backbone, and at least one group of dyes comprising a first dye and a second dye attached to the nanostructure backbone. The first dye and the second dye are within a distance from each other that enables an energy transfer between the first dye and the second dye. One of the dyes of the group of dyes is configured to emit light upon the energy transfer.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 shows a schematic view of a nanostructure backbone of a label according to some embodiments;

FIG. 2 shows a schematic view of groups of dyes on the nanostructure backbone according to some embodiments;

FIG. 3 shows a schematic view of a maker comprising a planar nanostructure backbone with groups of dyes according to some embodiments;

FIG. 4 shows a schematic view of attachments of dyes to the nanostructure backbone according to some embodiments;

FIG. 5 shows a schematic view of a comparison of the groups of dyes and individual dyes on the nanostructure backbone according to some embodiments; and

FIG. 6 shows a schematic view of a marker comprising a linear nanostructure backbone with the groups of dyes and further groups of dyes according to some embodiments.

### DETAILED DESCRIPTION

Embodiments of the present invention provide a label for usage in a marker for marking a target molecule in a biological sample, which enable distinguishing between a large number of target molecules.

In a first aspect, a label is provided for usage in a marker for marking a target molecule in a biological sample. The label comprises a nanostructure backbone, at least one group of dyes comprising a first dye and a second dye attached to the nanostructure backbone, wherein the first dye and the second dye are within a distance (D) from each other that enables an energy transfer between the first dye and the second dye, and wherein one of the dyes of the group of dyes is configured to emit light upon the energy transfer. In case there are two dyes in the group of dyes, one of the dyes of the group of dyes might be configured or selected such that it acts as a donor and the other dye of the group of dyes might be configured or selected such that it acts as an acceptor of the energy transfer. The acceptor may be configured to emit light as a result of the energy transfer. The energy transfer may be a fluorescence resonance energy transfer (FRET). The fluorescence lifetime of the light emission of the dye is shorter when the emission is caused by the energy transfer compared to emission caused by direct excitation at an excitation wavelength of the same dye. Thus, the emission of the dye may at least be characterised by whether the emission is caused by direct excitation or by the energy transfer. This enables providing a diverse set of labels that may be distinguished from each other not only by their emission wavelengths, but also by their fluorescence lifetime or by a measurement result depending on the fluorescence lifetime. In turn this allows distinguishing between a large set of different target molecules.

Moreover, the number of different labels that may be provided from the at least two dyes is increased by the label according to embodiments of the invention. From two dyes a plurality of distinguishable fluorescent labels may be generated, first, a label with only the FRET donor (first) dye, second, a label with only the FRET acceptor (second) dye, third, a label with both (first and second) dyes of the group of dyes where the dyes are at a distance large enough to not form the FRET pair, or, fourth, the label with both (first and second) dyes of the group of dyes forming the FRET pair. As these can all be differentiated at least by their excitation, emission and/or fluorescence lifetime due to the fact that FRET leads to a shortening of fluorescence lifetime of the donor, the number of discernible labels is always greater than the number of dyes used to generate the labels, as long as at least one group of dyes can be found within the plurality of dyes that allows the energy transfer between them.

Preferably, the distance (D) between the first dye and the second dye is in a range between 1 nm and 10 nm. This enables particular efficient energy transfer between the dyes of the group of dyes.

It is preferred, that the nanostructure backbone comprises nucleic acids. For example, the nanostructure backbone may comprise DNA, RNA and/or LNA. In particular, the nanostructure backbone consists of nucleic acids in the form of oligonucleotides. This enables easy and reproducible synthesis and assembly of the backbone.

In a preferred embodiment, the nanostructure backbone is a DNA origami structure. DNA origami may be designed to provide a self-assembly nanostructure backbone of a particular predetermined shape. This enables an easy and reproducible synthesis and assembly of the backbone.

Preferably, the DNA origami structure comprises at least one scaffold strand and multiple staple strands, wherein the staple strands are complementary to at least parts of the scaffold strand and configured to bring the scaffold strand into a predetermined conformation. In particular, the strands are oligonucleotides. This enables easy and reproducible synthesis and assembly of the backbone, in particular, in a variety of predetermined shapes.

For the fabrication of such DNA origami-based structures longer DNA molecules (scaffold strands) are folded at precisely identified positions by so called staple strands. The DNA origami may be designed to provide a self-assembly nanostructure backbone of a particular predetermined shape. This enables an easy and reproducible synthesis and assembly of the backbone.

Preferably, the largest spatial extent of the nanostructure backbone is in a range from 1 nm to 10000 nm, preferably in a range from 5 nm to 500 nm. This enables easy introduction into the biological sample.

In a preferred embodiment, the nanostructure backbone has a linear shape, a planar shape, or a polyhedral shape. This enables a particular flexible use of the label. In particular, the shape of the nanostructure backbone may be chosen to specifically influence fluorescence properties of the dyes attached to the backbone. Besides the dyes being attached to the nanostructure backbone which might be used for an optical identification of the respective label or the marker, the shape of the nanostructure backbone to which the dyes are attached, respectively, might be used as an optical identification as well, if markers with labels comprising different shapes of the nanostructure backbones are applied in the same biological sample or experiment. As long as there are enough dyes attached to the respective nanostructure backbone, the shape of the nanostructure backbone may be determined as well, in order to e.g. differentiate the shape of a linear versus a circular nanostructure backbone.

Preferably, the first dye and the second dye are attached to one of the staple strands. In particular, the dyes are attached to the same staple strand at predetermined positions. Staple strands allow the spatially precise functionalisation of the DNA origami at their respective locations on the DNA origami. This enables precise and predictable positioning of dyes on the nanostructure backbone.

As mentioned, staple strands may be position-selectively functionalised. The positional resolution in this case is limited by the size of a nucleotide, which is in the range of a nanometre or below. This has been exploited in the prior art to generate fluorescent standards, wherein fluorescent dyes are connected to precisely located bands on the DNA origami. These standards are known as “nanoruler” and are used for the calibration of imaging systems like confocal or super resolution microscopes (e.g. STED), for example, as disclosed by US2014/0057805 A1.

Preferably, the dyes are covalently bound to the nanostructure backbone, or each dye comprises a specific oligonucleotide and the nanostructure backbone comprises complementary oligonucleotides, each complementary oligonucleotide configured to hybridise to one of the specific oligonucleotides of the dyes. Thus, each dye has a specific oligonucleotide to bind to a corresponding or complementary specific part of the nanostructure backbone. The complementary oligonucleotide may be part of the nanostructure backbone, preferably the staple strands, in particular when the nanostructure backbone comprises nucleic acids. This enables easy attachment of the dyes to the nanostructure backbone. The attachment of the dyes to the nanostructure backbone may also be called a linker.

Therefore, the DNA origami provides a scaffold for the dyes. Preferably, the DNA origami structure comprises at least one scaffold strand and multiple staple strands, wherein the staple strands are complementary to at least parts of the scaffold strand and configured to bring the scaffold strand into a predetermined conformation. In particular, the strands are oligonucleotides. This enables generating nanostructure backbones with predetermined two- or three-dimensional shapes that can self-assemble. Further, this enables the site-specific placement of dyes on the backbone. Preferably, the dyes may be attached to staple strands of the nanostructure backbone at predetermined positions. Staple strands allow the spatially precise functionalisation of the DNA origami at their respective locations on the DNA origami. Thus, each dye may be located along the nanostructure backbone at a particular staple strand or group of staple strands that are in close proximity. Since the staple strands are located at predetermined positions the positions of the dyes may equally be predetermined.

Further examples of directly attaching or linking the dyes to the staple strands, which mediate a position selective attachment to the nanostructure backbone, include direct chemical coupling through for example click chemistry reactions (e.g. Azide-Alkine) or through high-affinity interactions such as biotin-Streptavidin. In the latter case biotinylated staple strands and streptavidin-conjugated dyes may be used.

In a preferred embodiment, one of the dyes of the group of dyes is configured to emit emission light in response to excitation of the other dye of the group of dyes with excitation light. The emission of the one of the dyes is caused by the energy transfer from the other dye.

It is preferred that the first dye and/or the second dye are fluorophores.

Preferably, the first dye and the second dye have different excitation wavelengths and/or emission wavelengths. This enables optically distinguishing between the first and the second dye.

Preferably, a fluorescence lifetime of one of the dyes of the group of dyes differs depending on whether the respective fluorescence emission is caused by excitation at a particular excitation wavelength of the dye or caused by an energy transfer from the other dye of the group of dyes. In particular, the fluorescence lifetime of a particular one of the dyes is shorter when the emission is caused by the energy transfer. Thus, the emission of the dye may be characterised by at least two parameters, the fluorescent emission wavelength and the fluorescence lifetime. In particular, the emission of the dye caused by the energy transfer may be distinguished from the emission of the same dye caused by excitation at the excitation wavelength only by the different fluorescence lifetimes.

In a preferred embodiment, the label comprises a plurality of the group of dyes. This enables providing a label with bright fluorescence.

In a preferred embodiment, at least the group of dyes, comprising the first and the second dye, further comprises a further dye, wherein at least one of the first dye and/or the second dye are within a distance D′ of the further dye that enables an energy transfer between the first dye or second dye and the further dye. It is preferred that the distance D′ is in a range from 1 to 10 nm. In this embodiment, the energy transfer may occur upon excitation of the first dye between the first dye and the second dye and subsequently between the second dye and the further dye. Only the further dye emits emission light upon the energy transfer, in particular if the distance between the first dye and the second dye is smaller than a distance that enables an energy transfer between the first dye and the second dye, and if the distance between the second dye and the further dye is smaller than a distance that enables an energy transfer between the second dye and the further dye, and if the distance between the first dye and the further dye is larger than a distance that enables an energy transfer between the first dye and the further dye. This enables providing labels with diverse optical properties.

Preferably, the label further comprises at least one further group of dyes comprising a third dye and a fourth dye or at least two further dyes having characteristics being different to the first, second, third and/or fourth dye, respectively. It is preferred, that the dyes of the further group have different excitation wavelengths and/or emission wavelengths than the dyes of the group of dyes. This enables providing labels with diverse optical properties. This is beneficial when it is desirable to distinguish a large number of labels.

Preferably, the group of dyes and the further group of dyes are spaced apart from each other by a distance in a range from 5 nm to 5000 nm, preferably in a range from 10 nm to 500 nm, more preferably in a range from 10 to 50 nm, most preferably in a range from 10 to 20 nm. In particular, the group of dyes is at a distance from the further group of dyes such that an interaction, for example, quenching or an energy transfer, between the dyes of the group of dyes and the dyes of the further group of dyes is reduced to a minimum or prevented.

Preferably, the affinity reagent is one of an antibody, an antibody fragment, an oligonucleotide, an aptamer, a peptide, a drug, or a toxin.

The marker has the same advantages as the label described above. In particular, the marker may be supplemented using the features of the dependent claims directed at the label.

Embodiments of the invention are described with reference to the accompanying drawings.

### TERMS

In the sense of this document the following terms are used in the following way:

“Sample”: In the sense of this document “sample” refers to a “biological sample” which may also be named a biological specimen including, for example blood, serum, plasma, tissue, bodily fluids (e.g. lymph, saliva, semen, interstitial fluid, cerebrospinal fluid), feces, solid biopsy, liquid biopsy, explants, whole embryos (e.g. zebrafish, Drosophila), entire model organisms (e.g. zebrafish larvae, Drosophila embryos, C. elegans), cells (e.g. prokaryotes, eukaryotes, archea), multicellular organisms (e.g. Volvox), suspension cell cultures, monolayer cell cultures, 3D cell cultures (e.g. spheroids, tumoroids, organoids derived from various organs such as intestine, brain, heart, liver, etc.), a lysate of any of the aforementioned, a virus. In the sense of this document “sample” further refers to a volume surrounding a biological sample. Like for example in assays, where secreted proteins like growth factors, extracellular matrix constituents are being studied the extracellular environment surrounding a cell up to a certain assay-dependent distance, is also referred to as the “sample”. Specifically, affinity reagents brought into this surrounding volume are referred to in the sense of this document as being “introduced into the sample”.

“Affinity reagent”: In the sense of this document the term “affinity reagent” may in particular be an antibody, a single-domain antibody (also known as nanobody), a combination of at least two single-domain antibodies, an aptamer, an oligonucleotide, a morpholino, a PNA complementary to a predetermined RNA, DNA target sequence, a ligand (e.g. a drug or a drug-like molecule), or a toxin, e.g. Phalloidin a toxin that binds to an actin filament. In the sense of this document an affinity reagent is configured to bind a target molecule or to an analyte with a certain affinity and specificity such that it can be said that the affinity reagent is substantially specific to the target molecule or predetermined target structure. In the sense of this document “plurality of affinity reagents” contains the affinity reagents, which are configured to specifically bind to a predetermined target structure within the biological sample or to a predetermined chemical compound or to a predetermined chemical element or to an analyte. At least some of the affinity reagents from the plurality of affinity reagents are “introduced to the sample” such that the affinity reagents can attach to the respective predetermined target structure within the sample. In this context and in the sense of this document and as described above “introduced to the sample” may refer to being physically introduced into the volume of the sample or into a volume surrounding and assigned to the sample. An example of the latter case may be assays for secreted molecules for instance, which are best assessed in the extracellular space where they might be outside of the sample, but within a certain spatial context or vicinity of the sample.

“Predetermined target structure”, “target”, “analyte”: In the sense of this document “predetermined target structure” refers to a target molecule or a target structure or to an analyte, which may for example be a protein (e.g. a particular protein), an RNA sequence (e.g. the mRNA of a certain gene), a peptide (e.g. somatostatin), a DNA sequence (e.g. the a genetic locus or element), a metabolite (e.g. lactic acid), a hormone (e.g. estradiol), a neurotransmitter (e.g. dopamine), a vitamin (e.g. cobalamine), a micronutrient (e.g. biotin), a metal ion (e.g. metal and heavy metal ions like Cd(II), Co(II), Pb(II), Hg(II), U(VI)).

“Dye”: In the sense of this document the terms “fluorescent dye”, “fluorophore”, “fluorochrome”, “dye” are used interchangeably to denote a fluorescent chemical compound or structure and can be in particular one of the following: a fluorescent organic dye, a fluorescent quantum dot, a fluorescent dyad, a fluorescent carbon dot, graphene quantum dot or other carbon-based fluorescent nanostructure, a fluorescent protein, a fluorescent DNA origami-based nanostructure. From the organic fluorescent dyes in particular derivatives of the following are meant by the term “fluorescent dye”: xanthene (e.g. fluorescein, rhodamine, Oregon green, Texas), cyanine (e.g. cyanine, indocarbocyanine, oxacarbocyanine, thiacarbocyanine, merocyanine), derivatives, squaraine rotaxane derivatives, naphthalene, coumarin, oxadiazole, anthracene (anthraquinones, DRAQ5, DRAQ7, CyTRAK Orange), pyrene (cascade blue), oxazine (Nile red, Nile blue, cresyl violet, oxazine 170), acridine (proflavine, acridine orange, acridine yellow), arylmethine (auramine, crystal violet, malachite green), tetrapyrrole (porphin, phthalocyanine, bilirubin), dipyrromethene (BODIPY, aza-BODIPY), a phosphorescent dye, or a luminescent dye. The following trademark groups designated commercially available fluorescent dyes, which may include dyes belonging to different chemical families CF dye (Biotium), DRAQ and CyTRAK probes (BioStatus), BODIPY (Invitrogen), EverFluor (Setareh Biotech), Alexa Fluor (Invitrogen), Bella Fluore (Setareh Biotech), DyLight Fluor (Thermo Scientific), Atto and Tracy (Sigma-Aldrich), FluoProbes (Interchim), Abberior Dyes (Abberior Dyes), Dy and MegaStokes Dyes (Dyomics), Sulfo Cy dyes (Cyandye), HiLyte Fluor (AnaSpec), Seta, SeTau and Square Dyes (SETA BioMedicals), Quasar and Cal Fluor dyes (Biosearch Technologies), SureLight Dyes (Columbia Biosciences), Vio Dyes (Milteny Biotec) [list modified from: https://en.wikipedia.org/wiki/Fluorophore]. From the group of fluorescent proteins in particular the members of the green fluorescent protein (GFP) family including GFP and GFP-like proteins (e.g DsRed, TagRFP) and their (monomerized) derivatives (e.g., EBFP, ECFP, EYFP, Cerulaen, m Turquoise2, YFP, EYFP, mCitrine, Venus, YPet, Superfolder GFP, mCherry, mPlum) are meant by the term “fluorescent dye” in the sense of this document. Further from the group of fluorescent proteins the term “fluorescent dye” in the sense of this document may include fluorescent proteins, whose absorbance or emission characteristics change upon binding of ligand like for example BFPms1 or in response to changes in the environment like for example redox-sensitive roGFP or pH-sensitive variants. Further from the group of fluorescent proteins the term “fluorescent dye” in the sense of this document may include derivative of cyanobacterial phycobiliprotein small ultra red fluorescent protein smURFP as well as fluorescent protein nanoparticles that can be derived from smURFP. An overview of fluorescent proteins can be found in Rodriguez et al. 2017 in Trends Biochem Sci. 2017 February; 42 (2): 111-129. The term “fluorescent dye” in the sense of this document may further refer to a fluorescent quantum dot. The term “fluorescent dye” in the sense of this document may further refer to fluorescent carbon dot, a fluorescent graphene quantum dot, a fluorescent carbon-based nanostructure as described in Yan et al. 2019 in Microchimica Acta (2019) 186:583 and Iravani and Varma 2020 in Environ Chem Lett. 2020 Mar. 10:1-25. The term “fluorescent dye” in the sense of this document may further refer to a fluorescent polymer dot (Pdot) or nanodiamond. The term “fluorescent dye” in the sense of this document may further refer to a fluorescent dyad, like for example a dyad of a perylene antenna and a triangelium emitter as described in Kacenauskaite et al. 2021 J. Am. Chem. Soc. 2021, 143, 1377-1385.

The term “fluorescent dye” in the sense of this document may further refer to an organic dye, a dyad, a quantum dot, a polymer dot, a graphene dot, a carbon-based nanostructure, a DNA origami-based nanostructure, a nanoruler, a polymer bead with incorporated dyes, a fluorescent protein, an inorganic fluorescent dye, a SMILE (small molecule ionic isolation lattices), or a microcapsule filled with any of the aforementioned.

“Marker”: In the sense of this document “marker” is used to denote both a single molecule used as marker and a collection of identical molecules used as marker. A “marker” in the sense of this document is the combination of an affinity reagent configured to attach to a predetermined structure also referred to as a target molecule or an analyte and/or a “label”, which may directly conjugate to the affinity reagent or indirectly by one or more “linkers”.

“Label”: In the sense of this document the term label denotes an assembly of a backbone or supporting nanostructure, at least one dye, preferably at least one fluorescent dye. The label may have further functionalizations to allow connecting the label to an affinity reagent forming a marker, which can be used to mark an analyte.

“Linker”: In the sense of this document the linker denotes a unipartite chemical structure (e.g. a monomeric molecule or a polymer) or multipartite assembly of chemical structures linking a combination of fluorescent dyes to an affinity reagent. A linker might be directly or covalently coupled to the dyes and to the affinity reagent or indirectly through for example affinity tag-affinity ligand combination such as streptavidin-biotin interaction or a hapten or an oligonucleotide for example. In the case of covalent coupling this may be a site-selective coupling. Commonly used coupling chemistries such NHS-, maleimide, azide-alkine and a range of further so called click chemistries may be used to couple the linker to the affinity reagent and/or the linker to a dye. A linker may in particular comprise an oligonucleotide (e.g. DNA, RNA, LNA, PNA, morpholino, other artificial oligonucleotide), a peptide, a DNA-origami-based structure such as for example a nanoruler, a micro-/nanobead, a polymer, a micro-/nanocapsule, a micro-/nanocrystal, a carbontube, a carbon-based nanostructure (e.g. a graphene).

A linker may in particular comprise an oligonucleotide and another element of the group mentioned before, like for example comprise an oligonucleotide and a peptide.

“Readout device”: In the sense of this document “readout device” refers to a device used to perform fluorescence multicolour reading or imaging. A readout device typically includes at least one excitation light source, a detection system including at least one detection channel and may as well contain filters and/or dispersive optical elements such as prisms and/or gratings to route excitation light to the sample and/or to route emission light from the sample onto a detector or onto an appropriate area of the detector. The detection system in the sense of this document may comprise several detection channels, may be a spectral detector detecting multiple bands of the spectrum in parallel, or a hyperspectral detector detecting a contiguous part of the spectrum. The detection system comprises at least one detector, which may be a point-detector (e.g. a photomultiplier, an avalanche diode, a hybrid detector), an array-detector, a camera, hyperspectral camera. The detection system may record intensities per channel as is typically the case in cytometers or may be an imaging detection system that records images as in the case of plate readers or microscopes. A readout device with one detector channel, like for example a camera or a photomultiplier, may generate readouts with multiple detection channels using, for example, different excitation and emission bands. Readout devices allow a certain number of dyes to be analysed from a given biological sample in a given run. A “run” may refer to an “iteration” or “round”, i.e. the production of at least one readout for a given set of combinations of dyes and a given mapping of affinity reagents to combinations of dyes, wherein the affinity reagents are attached to the analytes. This number typically depends on the number of detection channels, n, the readout device is configured to provide, i.e. is able to spectrally resolve. In the case of microscopes the number of detection channels is typically 4 to 5 in the case of camera-based widefield detections (e.g. widefield epifluorescence microscopes, spinning disk microscopes, light sheet fluorescence microscopes), 5 to 12 detection channels in the case of microscopes with spectral detection concepts that typically rely on excitation or emission fingerprinting and (spectral/linear) unmixing. Hyperspectral imaging instead, which can differentiate a high number of dyes by providing very fine spectral resolution over a wide and contiguous spectral range is not yet widely deployed in microscopy. In addition to spectral properties also the lifetime of fluorescent dyes can be used to discern multiple dye species and differentiate them from autofluorescence effectively increasing the number of detection channels which might correspond also to the maximum number of dyes in a set (i.e. excitable by the same excitation light) that can be reliably separated.

“Oligonucleotide”: in the sense of this document refers to DNA, RNA, peptide nucleic acid, morpholino or locked nucleic acid, glycol nucleic acid, threose nucleic acid, hexitol nucleic acid or another form of artificial nucleic acid.

“Spot”: in the sense of this document refers to a volume in the sample or region surrounding the sample, which is being readout. The size and shape of spots is dictated by the effective point spread function of the imaging system used to acquire the data.

“Point spread function”: In the sense of this document the term “point spread function” is used to denote the main maximum of the point spread function and unless otherwise denoted the term refers to the effective point spread function (PSF) of the imaging system, which is generally elliptical, i.e. the lateral resolution is better than the axial resolution, but may approach an almost spherical shape as more views are acquired from preferably equidistant angles.

“Readout”: In the sense of this document the term “readout” refers to an image-based readout, which may be acquired on a microscope like a point-scanning confocal or a camera-based/widefield imaging system like for example a spinning disk microscope, a light sheet fluorescence microscope, a light field microscope, a stereomicroscope. Further the term “readout” refers to non-image based readouts like for example in a cytometer or a flow-through based readout device with at least one point detector or a line detector. A readout may consist of a discrete readout, like for example a single acquisition of an emission spectrum or image stack, a readout may be a readout data stream, like for example in a point-scanning confocal or cytometer, which is substantially continuous. Further a readout may be a sequence of images for example a spectral or hyperspectral image stack, wherein in each image fluorescence emission of different wavelength bands is recorded.

“Readout volume”: In the sense of this document the term “readout volume” refers to the volume which is effectively detected by an optical system such as a microscope or a cytometer at a given moment in time. For systems with a continuous data stream, the “readout volume” is determined by a clock like a “pixel clock”, which divides a continuous data stream into chunks that are then assigned to a certain time point or spatial location. The readout volume might depend on the effective point spread function of the imaging system, e.g. an effective point spread function might define or confine the maximum extent of a readout volume. The effective point spread function depends on the illumination and detection point spread functions of the imaging system and may be substantially similar like in the case of point-scanning confocal microscope or substantially different like in the case of a selective plane of illumination microscope for example.

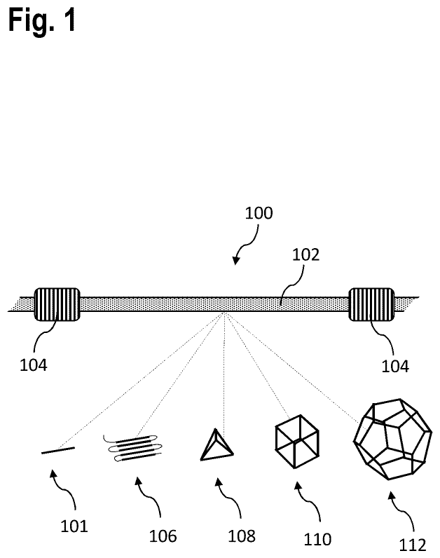

FIG. 1 shows a schematic view a part of a nanostructure backbone 100 of a label. The nanostructure backbone 100 has a linear shape 101. The nanostructure backbone 100 preferably comprises nucleic acids. In particular, the nanostructure backbone 100 is DNA-origami based, which allows generating arbitrary, stable two- and three-dimensional shapes. The DNA-origami based backbone 100 comprises at least one scaffold strand 102 and multiple staple strands 104 that hold together the scaffold strand 102 at predetermined positions to generate the linear shape 101 of the backbone 100. The respective strands 102, 104 are, for example, oligonucleotide strands. The staple strands 104 are complementary to at least parts of the scaffold strand 102 and when hybridised to the scaffold strand 102, bring the scaffold strand 102 into a predetermined conformation, in case of the nanostructure backbone 100 into the linear shape 101. Thus, the staple strands 104, in particular, define predictable positions along the nanostructure backbone 100 with specific predetermined oligonucleotide sequences at these positions.

Alternatively, the nanostructure backbones may have a different shape, such as a planar shape 106, or a polyhedral shape, for example a pyramidal shape 108, a cube or cuboid shape 110, or a dodecahedral shape 112.

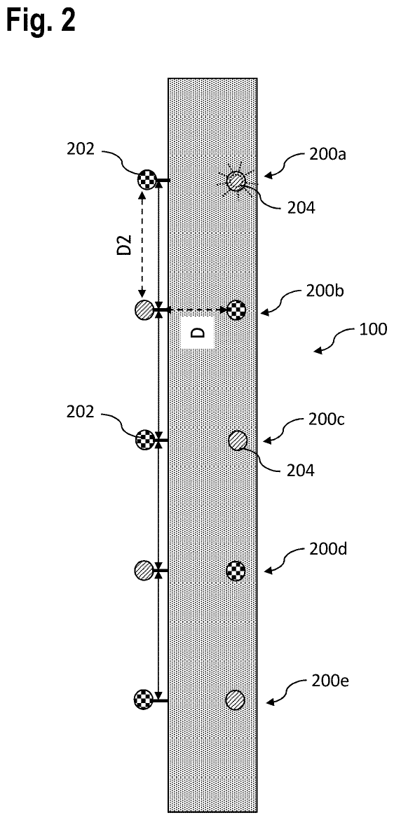

FIG. 2 shows part of the linear nanostructure backbone 100 with several groups 200a, 200b, 200c, 200d, 200e of dyes. Each group 200a-200e comprising a first dye 202 and a second dye 204. The dyes 202, 204 of each group 200a-200e are configured to enable an (radiation-free) energy transfer between the dyes 202, 204. In particular, a Förster resonance energy transfer or a fluorescence resonance energy transfer (FRET) where one of the dyes 202, 204 is a donor and the other one is an acceptor of the energy transfer. The efficiency of this transfer is inversely proportional to the distance between the donor and the acceptor. Preferably, the distance between the dyes 202, 204 is between 1 and 10 nm, specifically at 5 nm. In particular, the dyes 202, 204 are fluorophores.

The dyes 202, 204 are preferably attached to one of the staple strands 104 (not shown in FIG. 2) of the nanostructure backbone 100. Since the location of the staple strands 104 is predetermined when designing the DNA-origami of the nanostructure backbone 100, their (relative) location on the nanostructure backbone 100 is predetermined as well, enabling precise positioning of the dyes 202, 204 when they are attached to the staple strands 104. Preferably, the dyes 202, 204 of a group 200a-200e are attached to the nanostructure backbone 100 at a distance D between 1 nm to 10 nm from each other. In particular, this allows FRET between the dyes 202, 204 to occur when one of the dyes 202, 204 is excited with excitation light at a respective excitation wavelength. The energy transfer, for example FRET, from the excited donor dye, for example dye 202, to the acceptor dye, for example dye 204, of one of the groups of dyes 200a-200e causes the acceptor dye to emit fluorescence light at an emission wavelength.

The groups 200a-200e of dyes are spaced apart from each other by a distance D2 between 1 nm to 10 nm, or alternatively between 10 nm to 1000 nm, or preferably between 10 to 50 nm, most preferably between 10 to 20 nm. Specifically, only neighbouring groups 200a-200e are distanced from each other by the distance D2, but not all groups 200a-200e need to be distanced from all other groups 200a-200e by the distance D2.

Depending on the properties of the dyes in neighbouring groups smaller or larger distances between the groups may be desirable to generate combinatorial labels of minimal physical size that contain multiple groups of dyes, while avoiding that groups of dyes influence each other due to interactions, such as quenching or energy transfer.

Alternatively, a single group 200a-200e of dyes may be attached to the nanostructure backbone 100. However, providing several groups 200a-200e of dyes increases the brightness of the label.

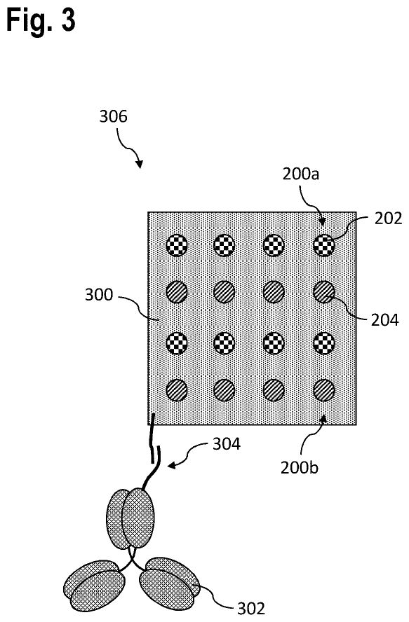

FIG. 3 shows a nanostructure backbone 300 having a planar or sheet-like shape with dyes 202, 204 attached. The nanostructure backbone 300 with the attached dyes 202, 204 may be called a label. One of the dyes 202 and one of the dyes 204 form groups 200a, 200b of dyes as described above. The dyes are attached to the nanostructure backbone 300 at a distance D of 2.5 nm. Similarly, the distance D2 between the groups of dyes 200a, 200b is 2.5 nm.

Further, the nanostructure backbone 300 is attached to an affinity reagent 302 via a linker 304. The affinity reagent 302 is an antibody. Alternatively, the affinity reagent 302 may be an antibody fragment, an oligonucleotide, an aptamer, a peptide, a drug or a toxin. In particular, the linker 304 may comprise two oligonucleotides that are partially complementary to each other. One of the oligonucleotides is attached to the nanostructure backbone 300 and the other one of the oligonucleotides is attached to the affinity reagent 302. The two oligonucleotides may hybridise at their complementary parts. The nanostructure backbone 300 with attached dyes 202, 204 and the affinity reagent 302 may also be termed a marker 306. In particular, the marker enables marking target molecules in biological samples. This means the affinity reagent 302 may specifically attach to the target molecules in the sample. With the nanostructure backbone 300 attached to the affinity reagent 302, the dyes 202, 204 enable optically identifying the position of the target molecules in the sample, for example, with an optical read-out device such as a microscope.

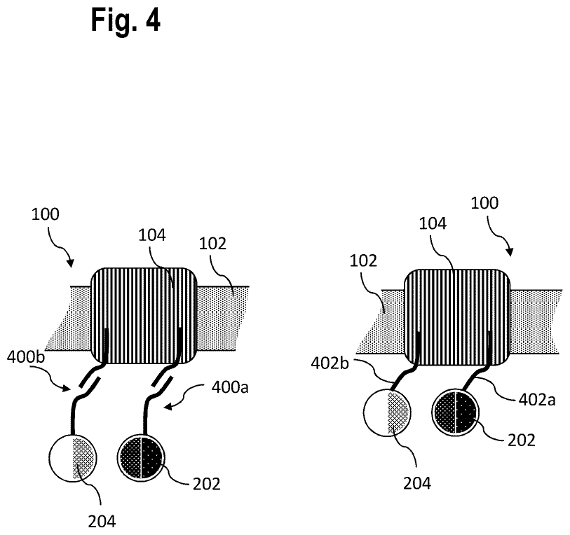

FIG. 4 shows embodiments of attachments of the dyes 202, 204 to the nanostructure backbone 100. The dyes 202, 204 may be attached to the nanostructure backbone 100 via linkers 400a, 400b. The linkers 400a, 400b each comprise two parts that may specifically bind to each other. For example, the linkers 400a, 400b may each comprise two oligonucleotides that are partially complementary to each other. One of the oligonucleotides is attached to the nanostructure backbone 100 and the other one of the oligonucleotides is attached to one of the dyes 202, 204. The two oligonucleotides may hybridise at their complementary parts in order to attach the respective dye 202, 204.

Alternatively, the dyes 202, 204 may be directly attached to the nanostructure backbone 100 via linkers 402a, 402b.

Moreover, the dyes are preferably attached at the staple strands 104 of the backbone 100, as shown in FIG. 4. This results in a predictable location of the dyes 202, 204 on the nanostructure backbone 100.

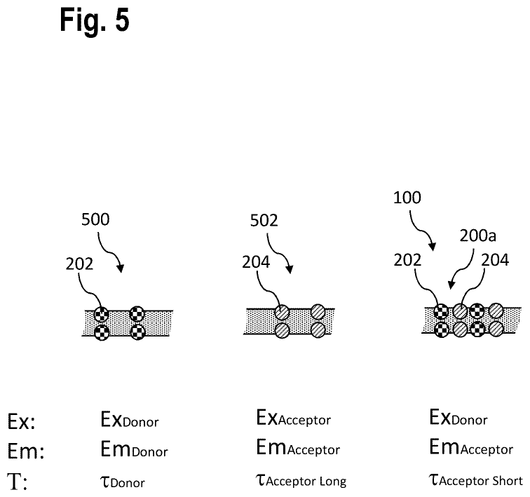

FIG. 5 schematically depicts a comparison of the group 200a-200e of dyes on the backbone 100 and the dyes 202, 204 individually on backbones 500, 502, respectively.

In comparison to the backbone 100, the backbone 500 only has dyes 202 attached, backbone 502 only has dyes 204 attached. When the dyes 202, 204 are each excited with their respective excitation light at a particular excitation wavelength of the dyes 202, 204, the dyes 202, 204 emit emission light at a particular emission wavelength. Preferably the dye 202 and the dye 204 differ from each other in their fluorescent properties such as excitation wavelength and/or emission wavelength. This enables distinguishing between the dyes 202, 204 and thus the associated backbones 500, 502 and labels, when optically reading them out with a fluorescent microscope, for example. Further, the individual dyes 202, 204 as attached to the backbones 500, 502 each have a particular fluorescent lifetime (t).

As described above, groups 200a of dyes are attached to the backbone 100 and thus it has dyes 202 attached that act as donors and dyes 204 that act as acceptors of an energy transfer, in particular FRET. When the donor dye 202 is excited with excitation light at the particular excitation wavelength of the dye 202, instead of emitting emission light, a FRET from the dye 202 to the dye 204 causes the dye 204 to emit emission light at the particular emission wavelength of the dye 204. Further, the emission of dye 204 caused by FRET generally has a shorter fluorescence lifetime (t) than the emission of dye 204 caused by direct excitation of dye 204 with excitation light. Thus, the fluorescence lifetime of the dye 204 may be used as a further parameter and in addition to excitation and emission wavelengths to reliably distinguish between labels using the same set of dyes 202, 204. Specifically, to distinguish between labels comprising the nanostructure backbone 100, 500, 502, for example. This enables generating a larger number of distinguishable labels using the same number of dyes 202, 204.

DNA origami-based nanostructures are suited to generate labels from a plurality of fluorescent dyes, whose members form a plurality of groups of dyes. This allows the generation of a plurality of distinguishable fluorescent labels, which contain first, only the FRET donor dye 202, second, only the FRET acceptor dye 204, or, third, both dyes 202, 204 of the group 200a-200e of dyes. As these can be differentiated by their excitation (Ex, ExDonor, ExAcceptor), emission (Em, EmDonor, EmAcceptor), and fluorescence lifetime (t, tDonor, tAcceptor Long, tAcceptor Short), due to the fact that FRET leads to a shortening of fluorescence lifetime, the number of discernible labels is always greater than the number of dyes in the plurality of dyes so as long as at least one FRET group can be found within the plurality of dyes.

The fluorescence lifetime of the emission of dye 204 when caused by FRET is also influenced by the environment of the group 200a of dyes, for example, the geometry of the nanostructure backbone 100, 300, or the spatial arrangement of the dyes 202, 204 to each other. Thus, the environment may be specifically designed to influence the fluorescence lifetime of the FRET group.

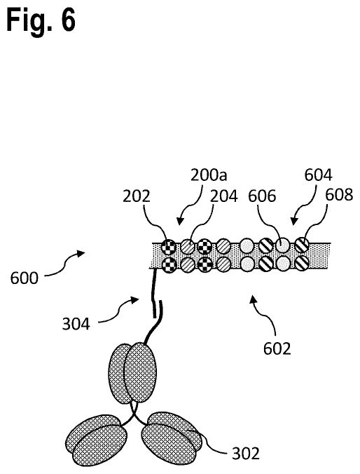

FIG. 6 shows a schematic view of a marker 600. The marker 600 comprises the affinity reagent 302 that attaches to a nanostructure backbone 602 via the linker 304. Attached to the nanostructure backbone 602 are groups 200a of the dyes 202, 204. In addition, further groups 604 of dyes 606, 608 are attached to the nanostructure backbone 602. Similar to the dyes 202, 204 of the group 200a, the dyes 606, 608 of the group 604 are configured to enable an energy transfer, for example FRET, between the dyes 606, 608. In particular, the dyes 606, 608 are attached to the nanostructure backbone 602 at a distance to each other in the range of 1 nm to 10 nm.

Furthermore, the dyes 606, 608 may have different excitation and/or emission wavelengths compared to the dyes 202, 204. In particular, the emission wavelength caused by the FRET acceptor is preferably different between the acceptor dyes of the groups 200a, 604.

Thus, the combination of several groups of dyes on the same nanostructure backbone enables generating a larger number of labels and consequently markers that can be distinguished from each other when optically read-out, for example, by a fluorescence microscope.

In an alternative embodiment, more than two groups of dyes may be attached to the nanostructure backbone 602. This enables generating a larger number of distinguishable labels.

As used herein the term “and/or” includes any and all combinations of one or more of the associated listed items and may be abbreviated as “/”.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE SIGNS

100, 300, 500, 502,
602 Nanostructure backbone
101, 106, 108, 110,
112 Shapes of nanostructure backbone
102 Scaffold strand
104 Staple strand
200a, 200b, 200c,
200d, 200e, 604 Group of dyes
202 First dye
204 Second dye
302 Affinity reagent
304 Affinity reagent linker
306, 600 Marker
400a, 400b Indirect dye linker
402a, 402b Direct dye linker
606 Third dye
608 Fourth dye

1. A label for usage in a marker for marking a target molecule in a biological sample, the label comprising:
a nanostructure backbone, and
at least one group of dyes comprising a first dye and a second dye attached to the nanostructure backbone,
wherein the first dye and the second dye are within a distance from each other that enables an energy transfer between the first dye and the second dye, and
wherein one of the dyes of the group of dyes is configured to emit light upon the energy transfer.

2. The label according to claim 1, wherein the distance between the first dye and the second dye is in a range between 1 nm and 10 nm.

3. The label according to claim 1, wherein the nanostructure backbone comprises nucleic acids.

4. The label according to claim 1, wherein the nanostructure backbone is comprises a DNA origami structure.

5. The label according to claim 4, wherein the DNA origami structure comprises at least one scaffold strand and multiple staple strands, wherein the staple strands are complementary to at least parts of the scaffold strand and configured to bring the scaffold strand into a predetermined conformation.

6. The label according to claim 1, wherein a largest spatial extent of the nanostructure backbone is in a range from 1 nm to 10000 nm.

7. The label according to claim 1, wherein the nanostructure backbone has a linear shape, a planar shape, or a polyhedral shape.

8. The label according to claim 5, wherein the first dye and the second dye are attached to one of the staple strands.

9. The label according to claim 1, wherein the dyes of the group of dyes are covalently bound to the nanostructure backbone, or
wherein each dye of the group of dyes comprises a specific oligonucleotide, and the nanostructure backbone comprises complementary oligonucleotides, each complementary oligonucleotide configured to hybridise to one of the specific oligonucleotides of the dyes.

10. The label according to claim 1, wherein one of the dyes of the group of dyes is configured to emit emission light in response to excitation of the other dye of the group of dyes with excitation light.

11. The label according to claim 1, wherein the first dye and the second dye have different excitation wavelengths and/or emission wavelengths.

12. The label according to claim 1, wherein a fluorescence lifetime of one of the dyes of the group of dyes differs depending on whether a respective fluorescence emission is caused by excitation at a particular excitation wavelength of the dye or caused by an energy transfer from the other dye of the group of dyes.

13. The label according to claim 1, comprising a plurality of groups of dyes.

14. The label according to claim 1, wherein at least the group of dyes further comprises a further dye, wherein at least one of the first dye or the second dye is within a second distance of the further dye that enables an energy transfer between the first dye and the further dye or between the second dye and the further dye.

15. The label according to claim 1, comprising at least one further group of dyes, the further group of dyes comprising a third dye and a fourth dye.

16. The label according to claim 15, wherein the group of dyes and the further group of dyes are spaced apart from each other by a second distance in a range from 5 nm to 5000 nm.

17. A marker for marking a target molecule in a biological sample, the marker comprising:
a label according to claim 1, and
an affinity reagent attached to the label and configured to specifically bind to the target molecule.

18. The marker according to claim 17, wherein the affinity reagent is one of an antibody, an antibody fragment, an oligonucleotide, an aptamer, a peptide, a drug, or a toxin.
